SEQUENCE LISTING (3 sequences)

SEQ ID NO: 1 - DNA, 1620 bp, Rattus norvegicus
cgtagtagcggaggcttagtccgcgcggcgctgcactgggattctcgcggtctggttcatctcccctttggcatctccttcttggcggccatgaagcggcagaacgtgcgtaccctgtccttgatcgcctgtaccttcacctacctgctggtgggtgccgcggtgttcgacgccctcgagtcggaccatgagatgcgcgaggaggagaaacttaaagcagaagaggtccgcctcagaggcaagtacaacatcagctccgatgactaccagcagctggagctggtaatcctgcagtctgagccccaccgcgctggtgtccagtggaagttcgccgggtccttctacttcgctatcactgtcatcacaactatcggatatggacatgctgcacctggaaccgatgctggcaaggccttctgtatgttctatgctgtgctgggtatccctctgacgctggttatgttccagagcctgggcgagcgcatgaacaccttcgtgcgctacctgctgaaacggatcaagaagtgctgtggcatgcgcaacactgaagtttctatggagaacatggtgaccgtcggcttcttttcttgcatgggcctcgtgccttgggcggctgccttttcccagtgcgaagattggagcttcttccacgcttactactactgcttcattacactgactactatagggttcggcgactttgtggccctgcaatccaagggtgccctgcagaggaagccattctacgtggccttcagcttcatgtatatcctggtgggcctgaccgtcatcggtgccttcctcaatcttgtggtcctgcgattcctgaccatgaataccgatgaagatcttctggagggagaagttgcgcagatacttgctggaaacccaagacgggtggttgtccgtgtgcctcagagtcgcaagaggcaccaccccatgtacttcctcaggaaatacggccgaaccctgtgctatctctgcttccctggtgccaactggggtgatgatgatgacgatgatgatgacgccgtcgagaatgtcgtagttactactcctgttcctcctgctgttgctgctgctgctgctgctgctactcctggtcccagtaccaggaatgtccgggctacagtccactcggtttcctgcagggttgaagagatccctccggacgtgctgaggaacacctacttccggtccccattcggcgccatccctcctggaatgcacacctgcggggaaaaccacaggctgcacatccgtcgcaagtccatctaagtgtggggagggaagtacacggaagaatcatttgtcatgcagatgtaagtttcattgtcccaactcctctctccctccttatttattattattctctttttttgtgcttacagtcatcatcattcctttctctcttccctcctctcctgggttcatttctttccgacctttccagccaggcagagctgtccaaagggcaaatagaggcccatcctctctgaagctcgcacctgagcatgaagcatggattccttcctttctccccaccagagttatgccttacatttctccccaccctgcctcctctctctggggtggctttcctaggacaggtgtgagaac

SEQ ID NO: 2 - DNA, 1125 bp, Homo sapiens
atgaagaggcagaacgtgcggactctgtccctcatcgtctgcaccttcacctacctgctggtgggcgccgccgtgttcgacgccctcgagtcggaccacgagatgcgcgaggaggagaaactcaaagccgaggagatccggatcaaggggaagtacaacatcagcagcgaggactaccggcagctggagctggtgatcctgcagtcggaaccgcaccgcgccggcgtccagtggaaattcgccggctccttctactttgcgatcacggtcatcaccaccataggttatgggcacgctgcacctggcaccgatgcgggcaaggccttctgcatgttctacgccgtgctgggcatcccgctgacactggtcatgttccagagcctgggcgagcgcatgaacaccttcgtgcgctacctgctgaagcgcattaagaagtgctgtggcatgcgcaacactgacgtgtctatggagaacatggtgactgtgggcttcttctcctgcatggggacgctgtgcatcggggcggccgccttctcccagtgtgaggagtggagcttcttccacgcctactactactgcttcatcacgttgactaccattgggttcggggactacgtggccctgcagaccaagggtgccctgcagaagaagccgctctacgtggcctttagctttatgtatatcctggtggggctgacggtcatcggggccttcctcaacctggtcgtcctcaggttcttgaccatgaacagtgaggatgagcggcgggatgctgaagagagggcatccctcgccggaaaccgcaacagcatggtcattcacatccctgaggagccgcggcccagccggcccaggtacaaggcggacgtcccggacctgcagtctgtgtgctcctgcacctgctaccgctcgcaggactatggcggccgctcggtggcaccgcagaactccttcagcgccaagcttgccccccactacttccactccatctcttacaagatcgaggagatctcaccaagcacattaaaaaacagcctcttcccatcgcctattagctccatctctcctgggttacacagctttaccgaccaccagaggctgatgaaacgccggaagtccgtttag

SEQ ID NO: 3 - DNA, 18 bp, Artificial Sequence
  Oligonucleotide antisense
atggcttcctctgcaggg

(19) United States
(12) Patent Application Publication
(10) Pub. No.: US 2006/0246432 A1
(43) Pub. Date: Nov. 2, 2006

(54) Methods and materials for modulating task-3

(76) Inventors: Samuel J. Shuster, Edina, MN (US); Laura S. Stone, St. Louis Park, MN (US); Hong-Yan Zhang, Sundyberg (SE); Lucy Vulchanova Hart, St. Paul, MN (US)

Correspondence Address: FISH & RICHARDSON P.C., PO BOX 1022, MINNEAPOLIS, MN, 55440-1022, US

(21) Appl. No.: 10/500,444

(22) PCT Filed: Dec. 31, 2002
(86) PCT No.: PCT/US02/41834

§ 371 Date: May 18, 2005

Related U.S. Application Data
(60) Provisional application No. 60/346,070, filed on Dec. 31, 2001.

(51) Int. Cl. A61K 48/00; C12Q 1/68; C07H 21/02

(52) U.S. Cl. 435/6; 514/44; 536/23.1

(57) ABSTRACT

The invention relates to antisense oligonucleotides, compositions and methods useful for modulating the expression of TASK-3. The compositions comprise antisense oligonucleotides targeted to nucleic acids encoding TASK-3.

### TECHNICAL FIELD

This invention relates to antisense oligonucleotides targeted to specific nucleotide sequences. In particular, the invention pertains to antisense oligonucleotides targeted to the nucleic acid encoding TASK-3, and to their use for reducing cellular levels of TASK-3.

### BACKGROUND

TASK-3 is an outwardly rectifying acid-sensitive potassium channel that is regulated by extracellular pH. Potassium channels are protein complexes that form K⁺ selective pores in biological membranes. Potassium channels are thought to contribute to background currents that regulate resting membrane potential. Potassium channels also appear to be important for physiological functions associated with modifications of electrical membrane potential, such as hormone secretion and neuronal and muscular excitability. In addition, potassium channels appear to have a role in controlling K⁺ homeostasis and cell volume.

### SUMMARY

TASK-3 is mainly expressed in the central nervous system, and TASK-3 immunoreactivity is most apparent in the inner portion of lamina II in dorsal horn of spinal cord—an area implicated in nociception and chronic pain. Thus, TASK-3 may have a role in pain sensation. A method for reducing the level or activity of TASK-3 would be useful for modulating pain sensation in a subject suffering from chronic pain.

Antisense oligonucleotides can be targeted to specific nucleic acid molecules, and to thereby reduce expression of specific nucleic acid molecules. For example, antisense oligonucleotides targeted to TASK-3 mRNA could be used therapeutically to reduce the level of TASK-3 receptors in a patient suffering from chronic pain.

One challenge in generating useful antisense oligonucleotides is identifying nucleic acid segments within a target mRNA that are suitable targets for antisense molecules. Antisense oligonucleotides typically are targeted to segments within a target mRNA based on, for example, the function of those segments (e.g., translation start site, coding sequence, etc.). Such targeting approaches are often unsuccessful because they do not account for the tertiary structure of the specific mRNA target. Native mRNA generally is folded into a complex secondary and tertiary structure, rendering sequences on the interior of such folded molecules inaccessible to antisense oligonucleotides. Only antisense molecules directed to accessible portions of a native mRNA could effectively hybridize to the mRNA and potentially bring about a desired result. Therefore, TASK-3 antisense molecules useful to reduce levels of TASK-3 and alleviate pain should be targeted to accessible mRNA sequences.

The invention features TASK-3 antisense oligonucleotides targeted to accessible portions of TASK-3 mRNA. Such antisense oligonucleotides can be used therapeutically to reduce TASK-3 levels. The invention provides isolated antisense oligonucleotides that specifically hybridize within an accessible region of TASK-3 mRNA in its native form, wherein the antisense oligonucleotides inhibit production of TASK-3. The invention also provides methods for decreasing production of TASK-3 in cells or tissues. The method involves contacting cells or tissues with an antisense oligonucleotide that specifically hybridizes within an accessible region of TASK-3 mRNA.

The invention features isolated antisense oligonucleotide consisting essentially of 10 to 50 nucleotides. The oligonucleotides can specifically hybridize within an accessible region of TASK-3 mRNA in its native state, wherein said region is defined by nucleotides 99 through 112, 149 through 166, 197 through 207, 217 through 236, 290 through 300, 314 through 327, 448 through 462, 526 through 539, 710 through 742, 852 through 868, 896 through 910, 996 through 1028, 1042 through 1054, 1170 through 1183, or 1278 through 1296 of SEQ ID NO:1. The oligonucleotides can inhibit the production of TASK-3.

The invention also features an isolated antisense oligonucleotide consisting essentially of 10 to 50 nucleotides, wherein the oligonucleotide specifically hybridizes within an accessible region of TASK-3 mRNA, wherein the region is defined by nucleotides 55 through 70, 101 through 156, 163 through 194, 226 through 240, 305 through 322, 434 through 443, 481 through 489, 500 through 512, 515 through 524, 540 through 557, 595 through 615, 641 through 658, 685 through 696, 700 through 711, 775 through 786, 791 through 806, 829 through 837, 929 through 947, 998 through 1013, 1088 through 1102, or 1108 through 1116 of SEQ ID NO:2, and wherein said isolated antisense oligonucleotide inhibits the production of TASK-3.

Such an antisense oligonucleotide can include, for example, a modified backbone, one or more non-natural internucleoside linkages, one or more substituted sugar moieties, and one or more nucleotide base modifications or nucleotide base substitutions. Such an antisense oligonucleotide can be an oligonucleotide analog.

The invention also features compositions comprising such isolated antisense oligonucleotides. The compositions can include a plurality of isolated antisense oligonucleotides, wherein each antisense oligonucleotide specifically hybridizes within a different accessible region.

The invention also features a nucleic acid construct that includes a regulatory element operably linked to a nucleic acid encoding a transcript that specifically hybridizes within one or more accessible regions of TASK-3 mRNA in its native form. Host cell that contain such nucleic acids are also provided.

The invention features a method of identifying a compound that modulates pain ill a mammal. Such a method includes contacting cells comprising a TASK-3 nucleic acid with a compound; and detecting the amount of TASK-3 RNA or TASK-3 polypeptide in or secreted from the cell. Generally, a difference in the amount of TASK-3 RNA or TASK-3 polypeptide produced in the presence of the compound compared to the amount of TASK-3 RNA or TASK-3 polypeptide produced in the absence of the compound is an indication that the compound modulates pain in the mammal. The method can further include testing the compound in a mammal.

Typically, the amount of TASK-3 RNA is determined by Northern blotting, while the amount of TASK-3 polypeptide is determined by Western blotting. The compound can be an antisense oligonucleotide that specifically hybridizes within an accessible region of TASK-3 mRNA in its native form. The antisense oligonucleotide can inhibit production of TASK-3.

The invention also provides a method for modulating pain in a mammal. Such a method includes administering a compound that modulates the expression of TASK-3 to the mammal. Such a compound can be an antisense oligonucleotide that specifically hybridizes within an accessible region of TASK-3 mRNA in its native form. The antisense oligonucleotide can inhibit production of TASK-3. For example, the pain can be from diabetic neuropathy, postherpetic neuralgia, fibromyalgia, surgery, or chronic back pain.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is the nucleotide sequence of rat TASK-3 (SEQ ID NO:1). GenBank Accession No. NM053405.

FIG. 2 is the nucleotide sequence of human TASK-3 (SEQ ID NO:2). GenBank Accession No. NM016601.

FIGS. 3A, 3B, and 3C are line graphs depicting the effect of TASK-3 antisense treatment on neuropathic pain. FIG. 3A shows the response to thermal stimuli; FIG. 3B shows the response to mechanical stimuli; and FIG. 3C shows a compilation of the data expressed as percent reversal of the pain response over a range of doses of TASK-3 antisense oligonucleotides.

FIGS. 4A and 4B are line graphs depicting the percent reversal of hypersensitivity to thermal (FIG. 4A) and mechanical (FIG. 4B) stimuli following treatment with TASK-3 antisense oligonucleotides or random DNA oligomer controls.

### DETAILED DESCRIPTION

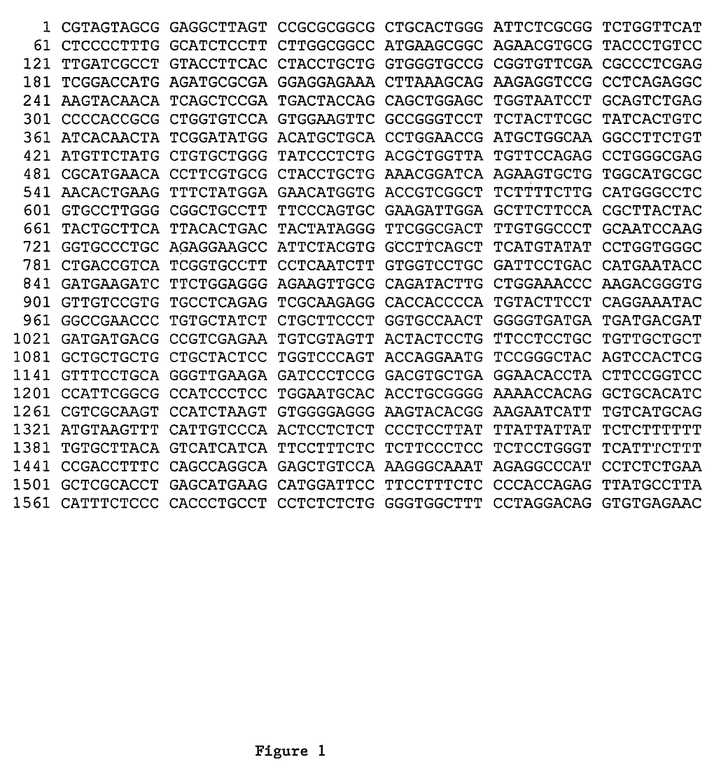

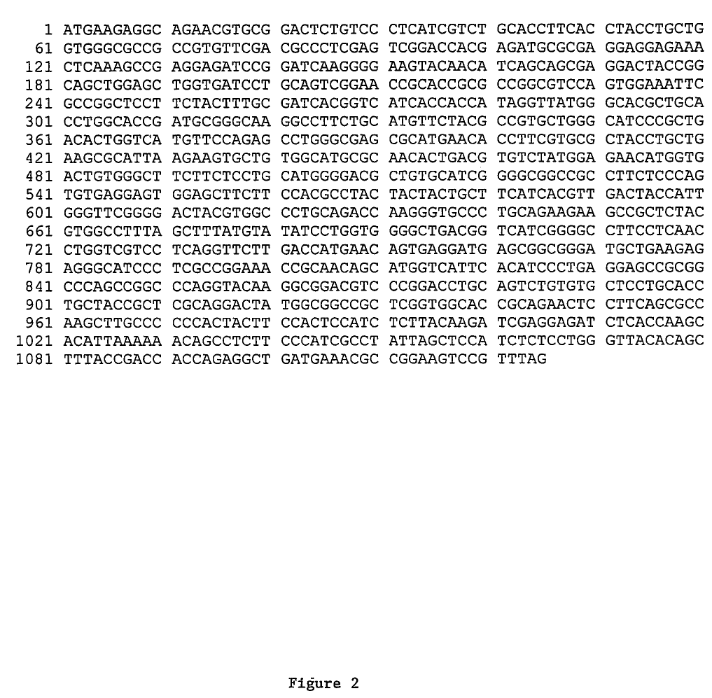

The present invention employs antisense molecules, particularly oligonucleotides, to modulate the function of target nucleic acid molecules. As used herein, the term “target nucleic acid” refers to both RNA and DNA, including cDNA, genomic DNA, and synthetic (e.g., chemically synthesized) DNA. The target nucleic acid can be double-stranded or single-stranded (i.e., a sense or an antisense single strand). In some embodiments, the target nucleic acid encodes a TASK-3 polypeptide. Thus, a “target nucleic acid” encompasses DNA encoding TASK-3, RNA (including pre-mRNA and mRNA) transcribed from such DNA, and also cDNA derived from such RNA. FIGS. 1 and 2 provide nucleic acid sequences that encode rat and human TASK-3 polypeptides, respectively (also designated as SEQ ID NO:1 and SEQ ID NO:2, respectively). An “antisense” molecule is a molecule containing nucleic acids or nucleic acid analogs that can specifically hybridize to a target nucleic acid. The modulation of function of a target nucleic acid by an antisense oligonucleotide is generally referred to as “antisense technology”.

The term “hybridization,” as used herein, means hydrogen bonding, which can be Watson-Crick, Hoogsteen, or reversed Hoogsteen hydrogen bonding, between complementary nucleoside or nucleotide bases. For example, adenine and thymine, and guanine and cytosine, respectively, are complementary nucleotide bases (often referred to in the art simply as “bases”) that pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides that can hydrogen bond with each other. Thus, “specifically hybridizable” is used to indicate a sufficient degree of complementarity or precise pairing such that stable and specific binding occurs between the oligonucleotide and the DNA or RNA target.

It is understood in the art that the sequence of an antisense oligonucleotide need not be 100% complementary to that of its target nucleic acid to be specifically hybridizable. An antisense oligonucleotide is specifically hybridizable when (a) binding of the oligonucleotide to the target DNA or RNA molecule interferes with the normal function of the target DNA or RNA, and (b) there is sufficient complementarity to avoid non-specific binding of the antisense oligonucleotide to non-target sequences under conditions in which specific binding is desired, i.e., under conditions in which in vitro assays are performed or under physiological conditions for in vivo assays or therapeutic uses.

Stringency conditions in vitro are dependent on temperature, time, and salt concentration (see, e.g., Sambrook et al., Molecular Cloning: A Laboratory Manual, Cold Spring Harbor Laboratory Press, NY, 1989). Typically, conditions of high to moderate stringency are used for specific hybridization in vitro, such that hybridization occurs between substantially similar nucleic acids, but not between dissimilar nucleic acids. Specific hybridization conditions are hybridization in 5×SSC (0.75 M sodium chloride/0.075 M sodium citrate) for 1 hour at 40° C. with shaking, followed by washing 10 times in 1×SSC at 40° C. and 5 times in 1×SSC at room temperature. Oligonucleotides that specifically hybridize to a target nucleic acid can be identified by recovering the oligonucleotides from the oligonucleotide/target hybridization duplexes (e.g., by boiling) and sequencing the recovered oligonucleotides.

In vivo hybridization conditions consist of intracellular conditions (e.g., physiological pH and intracellular ionic conditions) that govern the hybridization of antisense oligonucleotides with target sequences. In vivo conditions can be mimicked in vitro by relatively low stringency conditions, such as those used in the RiboTAG™ technology described below. For example, hybridization can be carried out in vitro in 2×SSC (0.3 M sodium chloride/0.03 M sodium citrate), 0.1% SDS at 37° C. Alternatively, a wash solution containing 4×SSC, 0.1% SDS can be used at 37° C., with a final wash in 1×SSC at 45° C.

The specific hybridization of an antisense molecule with its target nucleic acid can interfere with the normal function of the target nucleic acid. For a target DNA, antisense technology can disrupt replication and transcription. For a target RNA, antisense technology can disrupt, for example, translocation of the RNA to the site of protein translation, translation of protein from the RNA, splicing of the RNA to yield one or more mRNA species, and catalytic activity of the RNA. Antisense technology can also facilitate nucleolytic degradation of a target RNA. The overall effect of such interference with target nucleic acid function is, in the case of a nucleic acid encoding TASK-3, modulation of the expression of TASK-3. In the context of the present invention, “modulation” means a decrease in the expression of a gene and/or a decrease in cellular levels or activity of the protein encoded by a gene.

Identification of Target Sequences for TASK-3 Antisense Oligonucleotides

Antisense oligonucleotides preferably are directed at specific regions within a target nucleic acid. The process of “targeting” an antisense oligonucleotide typically begins with identifying a candidate target nucleic acid whose function is to be modulated. This nucleic acid can be, for example, a cellular gene (or mRNA transcribed from the gene) whose expression is associated with a particular disorder or disease state.

The targeting process also involves identifying a region or regions within a target nucleic acid where an antisense interaction can occur such that a desired effect is achieved. The desired effect can be, for example, modulation of TASK-3 expression or detection of TASK-3 mRNA (e.g., by using a detectably labeled antisense oligonucleotide). Antisense oligonucleotides have been directed at regions encompassing the translation initiation or termination codon of the open reading frame (ORF) of a gene. Antisense oligonucleotides have also been directed at ORFs, at the 5′ and 3′ untranslated regions of genes, and at intron regions and intron-exon junction regions.

Knowledge of the sequence and domain structure (e.g., the location of translation initiation codons, exons, or introns) of a target nucleic acid, however, is generally not sufficient to ensure that an antisense oligonucleotide directed to a specific region will effectively bind to and modulate the function of the target nucleic acid. In its native state, an mRNA molecule is folded into complex secondary and tertiary structures, and sequences on the interior of such folded structures generally are inaccessible to antisense oligonucleotides. For maximal effectiveness, antisense oligonucleotides can be directed to regions of a target mRNA that are most accessible, i.e., regions at or near the surface of a folded mRNA molecule.

Accessible regions of an mRNA molecule can be identified by, for example, the RiboTAG™ method, or mRNA Accessible Site Tagging (MAST), as described in PCT App. No. SE01/02054.

Using the RiboTAG™ method, oligonucleotides that can interact with a test mRNA in its native state (i.e., under physiological conditions) are selected and sequenced, thus leading to the identification of regions within the test mRNA that are accessible to antisense molecules. In a version of the RiboTAG™ protocol, the test mRNA is produced by in vitro transcription and is then immobilized, for example by covalent or non-covalent attachment to a bead or a surface (e.g., a magnetic bead). The immobilized test mRNA is then contacted by a population of oligonucleotides, wherein a portion of each oligonucleotide contains a different, random region. Oligonucleotides that can hybridize to the test mRNA under conditions of low stringency are separated from the remainder of the population (e.g. by precipitation of the magnetic beads). The selected oligonucleotides then can be amplified and sequenced; these steps of the protocol are facilitated if the random regions within each oligonucleotide are flanked on one or both sides by non-random regions that can serve as primer binding sites for PCR amplification.

In general, oligonucleotides useful for RiboTAG™ technology contain between 15 and 18 random bases, flanked on either side by non-random regions. These oligonucleotides are contacted by a test mRNA under conditions that do not disrupt the native structure of the mRNA (e.g., in the presence of medium pH buffering, salts that modulate annealing, and detergents and/or carrier molecules that minimize non-specific interactions). Typically, hybridization is carried out at 37 to 40° C., in a solution containing 1× to 5×SSC and about 0.1% SDS. Non-specific interactions can be further minimized by blocking the non-random sequence(s) in each oligonucleotide with the primers that will be used for PCR amplification of the selected oligonucleotides.

As described herein, accessible regions of the nucleic acids encoding human and rat TASK-3 have been mapped. Thus, antisense oligonucleotides of the invention can specifically hybridize within one or more accessible regions defined by: nucleotides 99 through 112, 149 through 166, 197 through 207, 217 through 236, 290 through 300, 314 through 327, 448 through 462, 526 through 539, 710 through 742, 852 through 868, 896 through 910, 996 through 1028, 1042 through 1054, 1170 through 1183, or 1278 through 1296 of SEQ ID NO:1.

Antisense oligonucleotides also can specifically hybridize within accessible regions defined by: nucleotides 55 through 70, 101 through 156, 163 through 194, 226 through 240, 305 through 322, 434 through 443, 481 through 489, 500 through 512, 515 through 524, 540 through 557, 595 through 615, 641 through 658, 685 through 696, 700 through 711, 775 through 786, 791 through 806, 829 through 837, 929 through 947, 998 through 1013, 1088 through 1102, or 1108 through 1116 of SEQ ID NO:2.

It should be noted that an antisense oligonucleotide may consist essentially of a nucleotide sequence that specifically hybridizes with an accessible region set out above. Such antisense oligonucleotides, however, may contain additional flanking sequences of 5 to 10 nucleotides at either end. Flanking sequences can include, for example, additional sequence of the target nucleic acid or primer sequence.

For maximal effectiveness, further criteria can be applied to the design of antisense oligonucleotides. Such criteria are known in the art, and are widely used, for example, in the design of oligonucleotide primers. These criteria include the lack of predicted secondary structure of a potential antisense oligonucleotide, an appropriate GC content (e.g., approximately 50%), and the absence of sequence motifs such as single nucleotide repeats (e.g., GGGG runs).

TASK-3 Antisense Oligonucleotides

Once one or more accessible target regions have been identified, antisense oligonucleotides sufficiently complementary to the target nucleic acid (i.e., that hybridize with sufficient strength and specificity to give the desired effect) can be synthesized. In the context of the present invention, the desired effect is the modulation of TASK-3 expression such that cellular TASK-3 levels are reduced. The effectiveness of an antisense oligonucleotide to modulate expression of a target nucleic acid can be evaluated by measuring levels of the mRNA or protein products of the target nucleic acid (e.g., by Northern blotting, RT-PCR, Western blotting, ELISA, or immunohistochemical staining).

In some embodiments, it may be useful to target multiple accessible regions of a target nucleic acid. In such embodiments, multiple antisense oligonucleotides can be used that each specifically hybridize to the same accessible region or to different accessible regions. Multiple antisense oligonucleotides can be used together or sequentially.

The antisense oligonucleotides in accordance with this invention preferably are from about 10 to about 50 nucleotides in length (e.g., 12 to 40, 14 to 30, or 15 to 25 nucleotides in length). Antisense oligonucleotides that are 15 to 23 nucleotides in length are particularly useful. However, an antisense oligonucleotide containing even fewer than 10 nucleotides (for example, a portion of one of the preferred antisense oligonucleotides) is understood to be included within the present invention so long as it demonstrates the desired activity of inhibiting expression of TASK-3.

An “antisense oligonucleotide” can be an oligonucleotide as described herein. The term “oligonucleotide” refers to an oligomer or polymer of ribonucleic acid (RNA) or deoxyribonucleic acid (DNA) or analogs thereof. This term includes oligonucleotides composed of naturally occurring nucleotide bases, sugars and covalent internucleoside (backbone) linkages, as well as oligonucleotides having non-naturally occurring portions which function similarly. Such modified or substituted oligonucleotides are often preferred over native forms because of desirable properties such as, for example, enhanced cellular uptake, enhanced affinity for a nucleic acid target, and increased stability in the presence of nucleases.

While antisense oligonucleotides are a preferred form of antisense molecules, the present invention includes other oligomeric antisense molecules, including but not limited to oligonucleotide analogs such as those described below. As is known in the art, a nucleoside is a base-sugar combination, wherein the base portion is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric molecule. The respective ends of this linear polymeric structure can be further joined to form a circular structure, although linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

TASK-3 antisense oligonucleotides that are useful in the present invention include oligonucleotides containing modified backbones or non-natural internucleoside linkages. As defined herein, oligonucleotides having modified backbones include those that have a phosphorus atom in the backbone and those that do not have a phosphorus atom in the backbone. For the purposes of this specification, and as sometimes referenced in the art, modified oligonucleotides that do not have a phosphorus atom in their internucleoside backbone also can be considered to be oligonucleotides.

Modified oligonucleotide backbones can include, for example, phosphorothioates, chiral phosphorothioates, phosphorodithioates, phosphotriesters, aminoalkylphosphotri-esters, methyl and other alkyl phosphonates (e.g., 3′-alkylene phosphonates and chiral phosphonates), phosphinates, phosphoramidates (e.g., 3′-amino phosphoramidate and aminoalkylphosphoramidates), thionophosphoramidates, thionoalkylphosphonates, thionoalkylphosphotriesters, and boranophosphates having normal 3′-5′ linkages, as well as 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. References that teach the preparation of such modified backbone oligonucleotides are provided, for example, in U.S. Pat. Nos. 4,469,863 and 5,750,666.

TASK-3 antisense molecules with modified oligonucleotide backbones that do not include a phosphorus atom therein can have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. References that teach the preparation of such modified backbone oligonucleotides are provided, for example, in U.S. Pat. Nos. 5,235,033 and 5,596,086.

In another embodiment, a TASK-3 antisense molecule can be an oligonucleotide analog, in which both the sugar and the internucleoside linkage (i.e., the backbone) of the nucleotide units are replaced with novel groups, while the base units are maintained for hybridization with an appropriate nucleic acid target. One such oligonucleotide analog that has been shown to have excellent hybridization properties is referred to as a peptide nucleic acid (PNA). In PNA molecules, the sugar-backbone of an oligonucleotide is replaced with an amide containing backbone (e.g., an aminoethylglycine backbone). The nucleotide bases are retained and are bound directly or indirectly to aza nitrogen atoms of the amide portion of the backbone. References that teach the preparation of such modified backbone oligonucleotides are provided, for example, in Nielsen et al., 1991, Science, 254:1497-1500, and in U.S. Pat. No. 5,539,082.

Other useful TASK-3 antisense oligonucleotides can have phosphorothioate backbones and oligonucleosides with heteroatom backbones, and in particular CH₂NHOCH₂, CH₂N(CH₃)OCH₂, CH₂ON(CH₃)CH₂, CH₂N(CH₃)N(CH₃)CH₂, and ON(CH₃)CH₂CH₂ (wherein the native phosphodiester backbone is represented as OPOCH₂) as taught in U.S. Pat. No. 5,489,677, and the amide backbones disclosed in U.S. Pat. No. 5,602,240.

Substituted sugar moieties also can be included in modified oligonucleotides. TASK-3 antisense oligonucleotides of the invention can comprise one or more of the following at the 2′ position: OH; F; O-, S-, or N-alkyl; O-, S-, or N-alkenyl; O-, S-, or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Useful modifications also can include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(C₂)ₙCH₃)]₂, where n and m are from 1 to about 10. In addition, oligonucleotides can comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, groups for improving the pharmacokinetic or pharmacodynamic properties of an oligonucleotide, and other substituents having similar properties. Other useful modifications include an alkoxyalkoxy group, e.g. 2′-methoxyethoxy (2′-OCH₂CH₂OCH₃), a dimethylaminooxyethoxy group (2′-O(CH₂)₂ON(CH₃)₂), or a dimethylamino-ethoxyethoxy group (2′-OCH₂OCH₂N(CH₂)₂). Other modifications can include 2′-methoxy (2′-OCH₃), 2′-aminopropoxy (2′-OCH₂CH₂CH₂NH₂), or 2′-fluoro (2′-F). Similar modifications also can be made at other positions within the oligonucleotide, such as the 3′ position of the sugar on the 3′ terminal nucleotide or in 2′-5′ linked oligonucleotides, and the 5′ position of the 5′ terminal nucleotide. Oligonucleotides also can have sugar mimetics such as cyclobutyl moieties in place of the pentofuranosyl group. References that teach the preparation of such substituted sugar moieties include U.S. Pat. Nos. 4,981,957 and 5,359,044.

Useful TASK-3 antisense oligonucleotides also can include nucleotide base modifications or substitutions. As used herein, “unmodified” or “natural” nucleotide bases include the purine bases adenine (A) and guanine (G), and the pyrimidine bases thymine (T), cytosine (C), and uracil (U). Modified nucleotide bases can include other synthetic and natural nucleotide bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleotide bases include those disclosed, for example, in U.S. Pat. No. 3,687,808.

Certain nucleotide base substitutions can be particularly useful for increasing the binding affinity of the antisense oligonucleotides of the invention. For example, 5-methylcytosine substitutions have been shown to increase nucleic acid duplex stability by 0.6 to 1.2° C. (Sanghvi et al., eds, Antisense Research aid Applications, pp. 276-278, CRC Press, Boca Raton, Fla., 1993). Other useful nucleotide base substitutions include 5-substituted pyrimidines, 6-azapyrimidines and N-2, N-6 and O-6 substituted purines such as 2-aminopropyladenine, 5-propynyluracil and 5-propynylcytosine.

Antisense oligonucleotides of the invention also can be modified by chemical linkage to one or more moieties or conjugates that enhance the activity, cellular distribution or cellular uptake of the oligonucleotide. Such moieties include but are not limited to lipid moieties (e.g., a cholesterol moiety); cholic acid; a thioether moiety (e.g., hexyl-S-tritylthiol); a thiocholesterol moiety; an aliphatic chain (e.g., dodecandiol or undecyl residues); a phospholipid moiety (e.g., di-hexadecyl-rac-glycerol or triethyl-ammonium 1,2-di-O-hexadecyl-rac-glycero-3-H-phosphonate); a polyamine or a polyethylene glycol chain; adamantane acetic acid; a palmityl moiety; or an octadecylamine or hexylamino-carbonyl-oxycholesterol moiety. The preparation of such oligonucleotide conjugates is disclosed in, for example, U.S. Pat. Nos. 5,218,105 and 5,214,136.

It is not necessary for all nucleotide base positions in a given antisense oligonucleotide to be uniformly modified. More than one of the aforementioned modifications can be incorporated into a single oligonucleotide or even at a single nucleoside within an oligonucleotide. The present invention also includes antisense oligonucleotides that are chimeric oligonucleotides. “Chimeric” antisense oligonucleotides can contain two or more chemically distinct regions, each made up of at least one monomer unit (e.g., a nucleotide in the case of an oligonucleotide). Chimeric oligonucleotides typically contain at least one region wherein the oligonucleotide is modified so as to confer, for example, increased resistance to nuclease degradation, increased cellular uptake, and/or increased affinity for the target nucleic acid. For example, a region of a chimeric oligonucleotide can serve as a substrate for an enzyme such as RNase H, which is capable of cleaving the RNA strand of an RNA:DNA duplex such as that formed between a target mRNA and an antisense oligonucleotide. Cleavage of such a duplex by RNase H, therefore, can greatly enhance the effectiveness of an antisense oligonucleotide.

Antisense molecules in accordance with the invention can include enzymatic ribonucleic acid molecules that can cleave other ribonucleic acid molecules (ribozymes). Antisense technologies involving ribozymes have shown great utility in research, diagnostic and therapeutic contexts. Methods for designing and using ribozymes are well known, and have been extensively described. Ribozymes in general are described, for example, in U.S. Pat. Nos. 5,254,678; 5,496,698; 5,525,468; and 5,616,459. U.S. Pat. Nos. 5,874,414 and 6,015,794 describe trans-splicing ribozymes. Hairpin ribozymes are described, for example, in U.S. Pat. Nos. 5,631,115; 5,631,359; 5,646,020; 5,837,855 and 6,022,962. U.S. Pat. No. 6,307,041 describes circular, hairpin, circular/hairpin, lariat, and hairpin-lariat hammerhead ribozymes. Ribozymes can include deoxyribonucleotides (see e.g., U.S. Pat. Nos. 5,652,094; 6,096,715 and 6,140,491). Such ribozymes are often referred to as (nucleozymes). Ribozymes can include modified ribonucleotides. Base-modified enzymatic nucleic acids are described, for example, in U.S. Pat. Nos. 5,672,511; 5,767,263; 5,879,938 and 5,891,684. U.S. Pat. No. 6,204,027 describes ribozymes having 2′-O substituted nucleotides in the flanking sequences. U.S. Pat. No. 5,545,729 describes stabilized ribozyme analogs. Other ribozymes having specialized properties have been described, for example, in U.S. Pat. No. 5,942,395 (describing chimeric ribozymes that include a snoRNA stabilizing motif), U.S. Pat. Nos. 6,265,167 and 5,908,779 (describing nuclear ribozymes), U.S. Pat. No. 5,994,124 (describing ribozyme-snRNA chimeric molecules having a catalytic activity for nuclear RNAs); and U.S. Pat. No. 5,650,502 (describing ribozyme analogs with rigid non-nucleotidic linkers).

The TASK-3 antisense oligonucleotides of the invention are synthesized in vitro and do not include antisense compositions of biological origin, except for oligonucleotides that comprise the subject antisense oligonucleotides and have been purified from or isolated from biological material. Antisense oligonucleotides used in accordance with this invention can be conveniently produced through the well-known technique of solid phase synthesis. Equipment for such synthesis is commercially available from several vendors including, for example, Applied Biosystems (Foster City, CA). Any other means for such synthesis known in the art additionally or alternatively can be employed. Similar techniques also can be used to prepare modified oligonucleotides such as phosphorothioates or alkylated derivatives.

Antisense Preparations and Methods for Use

The antisense oligonucleotides of the invention are useful for research (e.g., in developing assays to identify small molecule therapeutics), diagnostics, and for therapeutic use. For example, assays based on hybridization of antisense oligonucleotides to nucleic acids encoding TASK-3 can be used to evaluate levels of TASK-3 in a tissue sample. Hybridization of the antisense oligonucleotides of the invention with a nucleic acid encoding TASK-3 can be detected by means known in the art. Such means can include conjugation of an enzyme to the antisense oligonucleotide, radiolabeling of the antisense oligonucleotide, or any other suitable means of detection.

Those of skill in the art can harness the specificity and sensitivity of antisense technology for therapeutic use. Antisense oligonucleotides have been employed as therapeutic moieties in the treatment of disease states in animals, including humans. For therapeutic methods, the cells or tissues are typically within a vertebrate (e.g., a mammal such as a human).

The invention provides therapeutic methods for treating conditions involving abnormal expression (e.g., over-production) or altered function of TASK-3. By these methods, antisense oligonucleotides in accordance with the invention are administered to a subject (e.g., a human) suspected of having a disease or condition (e.g., chronic pain) that can be alleviated by modulating the expression of TASK-3. Typically, one or more antisense oligonucleotides can be administered to a subject suspected of having a disease or condition associated with the expression of TASK-3. The antisense oligonucleotide can be in a pharmaceutically acceptable carrier or excipient, and can be administered in amounts and for periods of time that will vary depending upon the nature of the particular disease, its severity, and the subject's overall condition. Typically, the antisense oligonucleotide is administered in an inhibitory amount (i.e., in an amount that is effective for inhibiting the production of TASK-3 in the cells or tissues contacted by the antisense oligonucleotides). The antisense oligonucleotides and methods of the invention also can be used prophylactically, e.g., to minimize pain in a subject that exhibits abnormal expression of TASK-3 or altered TASK-3 function.

The ability of a TASK-3 antisense oligonucleotide to inhibit expression and/or production of TASK-3 can be assessed, for example, by measuring levels of TASK-3 mRNA or protein in a subject before and after treatment. Methods for measuring mRNA and protein levels in tissues or biological samples are well known in the art. If the subject is a research animal, for example, TASK-3 levels in the brain can be assessed by in situ hybridization or immunostaining following euthanasia. Indirect methods can be used to evaluate the effectiveness of TASK-3 antisense oligonucleotides in live subjects. For example, reduced expression of TASK-3 can be inferred from reduced sensitivity to painful stimuli. As described in the Examples below, animal models can be used to study the development, maintenance, and relief of chronic neuropathic or inflammatory pain. Animals subjected to these models generally develop thermal hyperalgesia (i.e., an increased response to a stimulus that is normally painful) and/or allodynia (i.e., pain due to a stimulus that is not normally painful). Sensitivity to mechanical and thermal stimuli can be assessed (see Bennett, Methods in Pain Research, pp. 67-91, Kruger, Ed., 2001) to evaluate the effectiveness of TASK-3 antisense treatment.

Methods for formulating and subsequently administering therapeutic compositions are well known to those skilled in the art. Dosing is generally dependent on the severity and responsiveness of the disease state to be treated, with the course of treatment lasting from several days to several months, or until a cure is effected or a diminution of the disease state is achieved. Persons of ordinary skill in the art routinely determine optimum dosages, dosing methodologies and repetition rates. Optimum dosages can vary depending on the relative potency of individual oligonucleotides, and can generally be estimated based on EC₅₀ values found to be effective in in vitro and in vivo animal models. Typically, dosage is from 0.01 μg to 100 g per kg of body weight, and may be given once or more daily, weekly, or even less often. Dosage and dosing schedules vary depending on route of administration (e.g., systemic doses typically are greater than intrathecal or epidural doses). Following successful treatment, it may be desirable to have the patient undergo maintenance therapy to prevent the recurrence of the disease state.

The present invention provides pharmaceutical compositions and formulations that include the TASK-3 antisense oligonucleotides of the invention. TASK-3 antisense oligonucleotides therefore can be admixed, encapsulated, conjugated or otherwise associated with other molecules, molecular structures, or mixtures of oligonucleotides such as, for example, liposomes, receptor targeted molecules, or oral, rectal, topical or other formulations, for assisting in uptake, distribution and/or absorption.

A “pharmaceutically acceptable carrier” (also referred to herein as an “excipient”) is a pharmaceutically acceptable solvent, suspending agent, or any other pharmacologically inert vehicle for delivering one or more therapeutic molecules (e.g., TASK-3 antisense oligonucleotides) to a subject. Pharmaceutically acceptable carriers can be liquid or solid, and can be selected with the planned manner of administration in mind so as to provide for the desired bulk, consistency, and other pertinent transport and chemical properties, when combined with one or more of therapeutic molecules and any other components of a given pharmaceutical composition. Typical pharmaceutically acceptable carriers that do not deleteriously react with nucleic acids include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

The pharmaceutical compositions of the present invention can be administered by a number of methods depending upon whether local or systemic treatment is desired and depending upon the area to be treated. Administration can be, for example, topical (e.g., transdermal, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, antisense oligonucleotides can be administered by injection or infusion into the cerebrospinal fluid, preferably with one or more agents capable of promoting penetration of the antisense oligonucleotide across the blood-brain barrier.

Formulations for topical administration of antisense oligonucleotides include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Coated condoms, gloves and the like also may be useful. Conventional pharmaceutical carriers, aqueous, powder or oily bases, thickeners and the like may be necessary or desirable.

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders. Oligonucleotides with at least one 2′-O-methoxyethyl modification (described above) may be particularly useful for oral administration.

Compositions and formulations for parenteral, intrathecal or intraventricular administration can include sterile aqueous solutions, which also can contain buffers, diluents and other suitable additives (e.g., penetration enhancers, carrier molecules and other pharmaceutically acceptable carriers).

Pharmaceutical compositions of the present invention include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Liposomes are vesicles that have a membrane formed from a lipophilic material and an aqueous interior that can contain the antisense composition to be delivered. Liposomes can be particularly useful due to their specificity and the duration of action they offer from the standpoint of drug delivery. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene™ (Qiagen, Valencia, Calif.).

The TASK-3 antisense oligonucleotides of the invention further encompass any pharmaceutically acceptable salts, esters, or salts of such esters, or any other molecule which, upon administration to an animal including a human, is capable of providing (directly or indirectly) the biologically active metabolite or residue thereof. Accordingly, for example, the invention provides pharmaceutically acceptable salts of TASK-3 antisense oligonucleotides, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the oligonucleotides of the invention (i.e., salts that retain the desired biological activity of the parent oligonucleotide without imparting undesired toxicological effects). Examples of pharmaceutically acceptable salts of oligonucleotides include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); salts formed with organic acids (e.g., acetic acid, citric acid, oxalic acid, palmitic acid, or fumaric acid); and salts formed from elemental anions (e.g., chlorine, bromine, and iodine).

Pharmaceutical compositions containing the antisense oligonucleotides of the present invention also can incorporate penetration enhancers that promote the efficient delivery of nucleic acids, particularly oligonucleotides, to the skin of animals. Penetration enhancers can enhance the diffusion of both lipophilic and non-lipophilic drugs across cell membranes. Penetration enhancers can be classified as belonging to one of five broad categories, i.e., surfactants (e.g., sodium lauryl sulfate, polyoxyethylene-9-lauryl ether and polyoxyethylene-20-cetyl ether); fatty acids (e.g., oleic acid, lauric acid, myristic acid, palmitic acid, and stearic acid); bile salts (e.g., cholic acid, dehydrocholic acid, and deoxycholic acid); chelating agents (e.g., disodium ethylenediaminetetraacetate, citric acid, and salicylates); and non-chelating non-surfactants (e.g., unsaturated cyclic ureas).

Certain embodiments of the invention provide pharmaceutical compositions containing (a) one or more antisense oligonucleotides and (b) one or more other agents that function by a non-antisense mechanism. For example, anti-inflammatory drugs, including but not limited to non-steroidal anti-inflammatory drugs and corticosteroids, and antiviral drugs, including but not limited to ribivirin, vidarabine, acyclovir and ganciclovir, can be included in compositions of the invention. Other non-antisense agents (e.g., chemotherapeutic agents) are also within the scope of this invention. Such combined molecules can be used together or sequentially.

The antisense compositions of the present invention additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions of the present invention, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the antisense components within the compositions of the present invention. The formulations can be sterilized and, if desired, and the like which do not deleteriously interact with the nucleic acid(s) of the formulation.

The pharmaceutical formulations of the present invention, which can be presented conveniently in unit dosage form, can be prepared according to conventional techniques well known in the pharmaceutical industry. Such techniques include the step of bringing into association the active ingredients (e.g., the TASK-3 antisense oligonucleotides of the invention) with the desired pharmaceutical carrier(s) or excipient(s). Typically, the formulations can be prepared by uniformly and bringing the active ingredients into intimate association with liquid carriers or finely divided solid carriers or both, and then, if necessary, shaping the product. Formulations can be sterilized if desired, provided that the method of sterilization does not interfere with the effectiveness of the antisense oligonucleotide contained in the formulation.

The compositions of the present invention can be formulated into any of many possible dosage forms such as, but not limited to, tablets, capsules, liquid syrups, soft gels, suppositories, and enemas. The compositions of the present invention also can be formulated as suspensions in aqueous, non-aqueous or mixed media. Aqueous suspensions further can contain substances that increase the viscosity of the suspension including, for example, sodium carboxymethylcellulose, sorbitol, and/or dextran. Suspensions also can contain stabilizers.

Nucleic Acid Constructs

Nucleic acid constructs (e.g., a plasmid vector) are capable of transporting a nucleic acid into a host cell. Suitable host cells include prokaryotic or eukaryotic cells (e.g., bacterial cells such as E. coli, insect cells, yeast cells, and mammalian cells). Some constructs are capable of autonomously replicating in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and are replicated with the host genome.

Nucleic acid constructs can be, for example, plasmid vectors or viral vectors (e.g., replication defective retroviruses, adenoviruses, and adeno-associated viruses). Nucleic acid constructs include one or more regulatory sequences operably linked to the nucleic acid of interest (e.g., a nucleic acid encoding a transcript that specifically hybridizes to a TASK-3 mRNA in its native form). With respect to regulatory elements, “operably linked” means that the regulatory sequence and the nucleic acid of interest are positioned such that nucleotide sequence is transcribed (e.g., when the vector is introduced into the host cell).

Regulatory sequences include promoters, enhancers and other expression control elements (e.g., polyadenylation signals). See, e.g., Goeddel, Gene Expression Technology: Methods in Enzymology, 185, Academic Press, San Diego, Calif., 1990. Regulatory sequences include those that direct constitutive expression of a nucleotide sequence in many types of host cells and those that direct expression of the nucleotide sequence only in certain host cells (e.g., cell type or tissue-specific regulatory sequences) or at certain (e.g., developmental) times.

Articles of Manufacture

Antisense oligonucleotides of the invention can be combined with packaging material and sold as kits for reducing TASK-3 expression. Components and methods for producing articles of manufacture such as kits are well known. An article of manufacture may combine one or more of the antisense oligonucleotides set out in the above sections. In addition, the article of manufacture further may include buffers, hybridization reagents, or other control reagents for reducing or monitoring reduced TASK-3 expression. Instructions describing how the antisense oligonucleotides are effective for reducing TASK-3 expression can be included in such kits.

The invention will be further described in the following examples, which do not limit the scope of the invention described in the claims.

### EXAMPLES

### Example 1

### Materials and Methods

Determination of Accessible Sites within TASK-3 mRNA and Design of TASK-3 Antisense Oligonucleotides

Accessible regions of rat and human TASK-3 mRNA (as determined by the RiboTAG™ method) are shown in Table 1.

TABLE 1
Accessible sequences within TASK-3 mRNA

| | |
|---|---|
| Rat | Human |
| TASK-3 | TASK-3 |

| Start | End | Start | End |
|---|---|---|---|
| 99 | 112 | 55 | 70 |
| 149 | 166 | 101 | 156 |
| 197 | 207 | 163 | 194 |
| 217 | 236 | 226 | 240 |
| 290 | 300 | 305 | 322 |
| 314 | 327 | 434 | 443 |
| 448 | 462 | 481 | 489 |
| 526 | 539 | 500 | 512 |
| 710 | 742 | 515 | 524 |
| 852 | 868 | 540 | 557 |
| 896 | 910 | 595 | 615 |
| 996 | 1028 | 641 | 658 |
| 1042 | 1054 | 685 | 696 |
| 1170 | 1183 | 700 | 711 |
| 1278 | 1296 | 775 | 786 |
| | | 791 | 806 |
| | | 829 | 837 |
| | | 929 | 947 |
| | | 998 | 1013 |
| | | 1088 | 1102 |
| | | 1108 | 1116 |

Methods for Evaluating Pain in Rats Treated with Antisense TASK-3

Two different models of chronic pain were used to evaluate the effects of TASK-3 knock-down by intrathecally administered antisense oligonucleotides. Both models included the following six steps (described in greater detail below):

(1) spinal catheterization;

(2) nociceptive testing (baseline);

(3) induction of chronic neuropathic or inflammatory pain;

(4) nociceptive testing (post-injury);

(5) antisense injection; and

(6) nociceptive testing (post-treatment).

Spinal Catheterization: Male Sprague Dawley rats weighing between 200 and 250 g were obtained from Harlan (Indianapolis, Ind.). Rats were deeply anesthetized with a mixture containing 75 mg/kg ketamine, 5 mg/kg xylazine, and 1 mg/kg acepromazine, and a catheter (8.5 cm; PE-10) was passed to the lumbosacral intrathecal space through an incision in the dura over the atlantooccipital joint. Following surgery, animals were kept on a warming blanket and were periodically turned and carefully observed until completely recovered from anesthesia. Animals were allowed to recover for at least 3 days before being subjected to models of chronic pain.

Mechanical Nociceptive Testing: Baseline, post-injury, and post-treatment values for mechanical sensitivity were evaluated with calibrated monofilaments (von Frey filaments) according to the up-down method (Chaplan et al., 1994, J. Neurosci. Methods, 53:55-63). Animals were placed on a wire mesh platform and allowed to acclimate to their surroundings for a minimum of 10 minutes before testing. Filaments of increasing force were sequentially applied to the plantar surface of the paw just to the point of bending, and held for three seconds. The behavioral endpoint of the stimulus (achieved when the stimulus was of sufficient force) was the point at which the animal would lick, withdraw and/or shake the paw. The force or pressure required to cause a paw withdrawal was recorded as a measure of threshold to noxious mechanical stimuli for each hind-paw. The mean and standard error of the mean (SEM) were determined for each animal in each treatment group. The data were analyzed using repeated measures ANOVA followed by the Bonferonni post-hoc test. Since this stimulus is normally not considered painful and rats do not normally respond to filaments in the range selected, significant injury-induced increases in responsiveness in this test were interpreted as a measure of mechanical allodynia.

Thermal Nociceptive Testing: Baseline, post-injury, and post-treatment thermal sensitivities were determined by measuring withdrawal latencies in response to radiant heat stimuli delivered to the plantar surface of the hind-paws (Hargreaves et al., 1988, Pain, 32:77-88). Animals were placed on a plexiglass platform and allowed to acclimate for a minimum of 10 minutes. A radiant heat source was directed to the plantar surface, and the time to withdrawal was measured. For each paw, the withdrawal latency was determined by averaging three measurements separated by at least 5 minutes. The heating device was set to automatically shut off after a programmed period of time to avoid damage to the sldn of unresponsive animals. The data were analyzed using repeated measures ANOVA followed by the Bonferonni post-hoc test. Significant injury-induced increases in thermal response latencies were considered to be a measure of thermal hyperalgesia since the stimulus is normally in the noxious range.

Induction of Chronic Neuropathic Pain: The Spinal Nerve Ligation (SNL) model (Kim & Chung, 1992, Pain, 50:355-363) was used to induce chronic neuropathic pain. Rats were anesthetized with isoflurane, the L5 transverse process was removed, and the L5 and L6 spinal nerves were tightly ligated with 6-0 silk suture. The wound was then closed with internal sutures and external staples. All operations were performed on the left side.

Antisense Design and Injection: Antisense oligonucleotides were commercially synthesized (Midland Certified Reagent Company, Midland, Tex.) and purified prior to injection. Oligonucleotides were dissolved in dH₂O and delivered into the intrathecal space in a volume of 5 μl per injection as previously described (see, for example, Bilsky et al., 1996, Neurosci. Lett., 220:155-158; Bilsky et al., 1996, J. Pharmacol. Exp. Ther., 277:491-501; and Vanderah et al., 1994, Neuroreport., 5:2601-2605). Antisense oligonucleotides were administered twice daily for 3 to 4 days, beginning on the afternoon following post-injury (baseline) nociceptive testing. Antisense oligonucleotides that were used included the sequence ATG GCT TCC TCT GCA GGG (SEQ ID NO:3), which specifically hybridizes to nucleotides 725 through 742 of SEQ ID NO:1. Random oligonucleotides were used as controls.

Immunolocalization of TASK-3

Human spinal cord tissue was obtained post-mortem and immersion-fixed overnight in 4% paraformaldehyde. After fixation, the tissue was washed in phosphate buffered saline (PBS) for 2 to 3 days and stored in 10% sucrose solution. The spinal cord was sliced into 14 μm sections using a cryostat. Slide-mounted tissue sections were incubated in blocking buffer for 1 hour at room temperature, followed by incubation with primary antisera (guinea pig anti-TASK-3, 1:5000) overnight at 4° C. Staining was visualized using biotinylated tiramine amplification as previously described (Vulchanova et al., 1997, Neuropharm., 36:1229-42). For absorption control, the primary antisera were incubated with the corresponding peptide antigen (10 μg/ml) prior to application to tissue sections.

Human dorsal root ganglia were obtained post-mortem and frozen in liquid nitrogen. The frozen tissue was cut into 10 μm sections, which were thaw-mounted on cooled gelatin-coated slides. Sections were fixed with paraformaldehyde-picric acid fixative for 30 minutes immediately before processing for immunohistochemistry. The slide-mounted tissue sections were incubated in blocking buffer for 1 hour at room temperature, followed by incubation with primary antisera (guinea pig anti-TASK-3, 1:500) overnight at 4° C. Slides were washed three times in PBS, incubated with secondary antisera for 1 hour at room temperature, washed again and coverslipped. For absorption control, the primary antisera were incubated with the corresponding peptide antigen (10 μg/ml) prior to application to tissue sections. Staining was visualized with cyanine 3.18-conjugated secondary antisera (Jackson ImmunoResearch, West Grove, Calif.).

Immunolocalization assays as described above revealed that TASK-3 is localized in the dorsal horn of human spinal cord. TASK-3 was detected directly ventral to CGRP and substance P immunoreactive fibers, which suggests that TASK-3 is present in inner lamina II of the spinal cord. The spinal cord neurons in inner lamina II, as well as the sensory neurons that terminate there, reportedly play a significant role in chronic pain.

### Example 2

### Antisense Knockdown of TASK-3 in Rat Spinal Cord Supports a Role in Chronic Neuropathic Pain

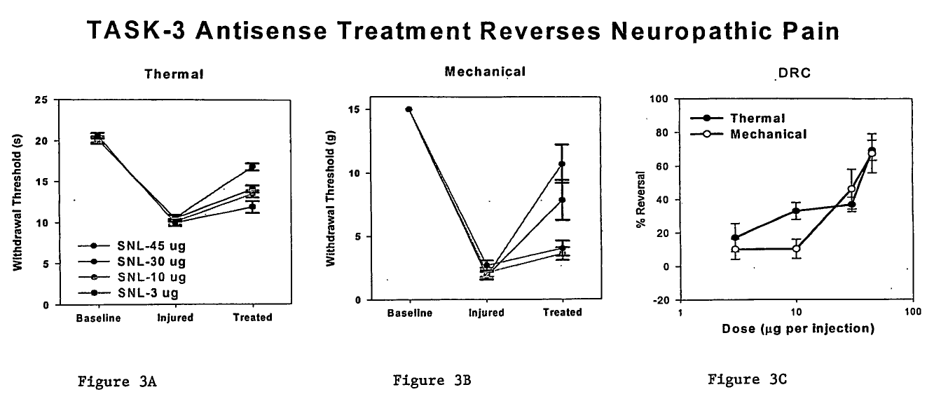

FIG. 3 demonstrates that treatment with a TASK-3 antisense oligonucleotide reversed the effects of chronic neuropathic pain in a dose dependent manner. Normal rats responded to a noxious heat stimulus with an average latency of 20 seconds. Following nerve injury, the response time decreased to about 10 seconds (FIG. 3A). Such a drop is analogous to the abnormal pain sensitivity observed in human patients suffering from chronic pain. Following three days of TASK-3 antisense treatment, there was a significant dose-related reversal of the nerve injury-induced hypersensitivity. FIG. 3B shows that TASK-3 antisense treatment also increased the tolerance to noxious mechanical stimuli. Normal animals rarely respond to stimuli of less than 15 g. Following nerve injury, however, animals withdrew from stimuli of only a few grams. TASK-3 antisense treatment reversed this hypersensitivity in a dose-dependent manner. The thermal and mechanical data are combined in FIG. 3C, which depicts the results as the percentage reversal of nerve injury-induced hypersensitivity as a function of antisense dose.

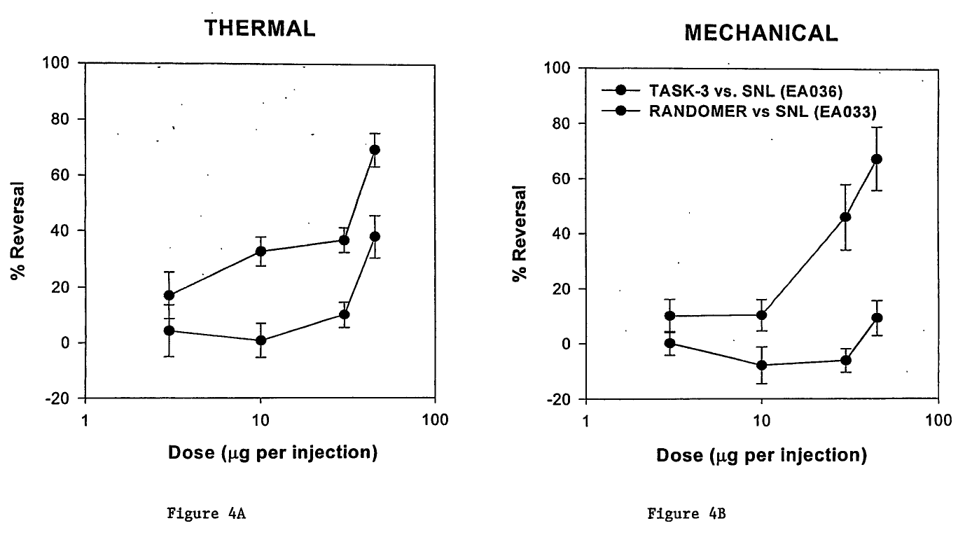

FIG. 4 shows that animals treated with random control oligonucleotides rather than the TASK-3 antisense oligonucleotide did not display a reversal of hypersensitivity to thermal (FIG. 4A) or mechanical (FIG. 4B) stimuli. Thus, the TASK-3 antisense oligonucleotide was significantly more effective for reducing pain.

### Example 3

### Quantitative TagMan RT-PCR Analysis of TASK-3 After Antisense Treatment

Quantitative PCR method is used to evaluate TASK-3 mRNA levels in control animals, and in animals with a chronic inflammation in one of the hindpaws, treated with TASK-3 antisense or a mismatch. Treatment with antisense reduces the level of TASK-3-mRNA in both inflamed and control animals.

TaqMan PCR is carried out using an ABI 7700 sequence detector (Perkin Elmer) on the cDNA samples. TaqMan primer and probe sets are designed from sequences in the GeneBank database using Primer Express (Perkin Elmer).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated antisense oligonucleotide consisting essentially of 10 to 50 nucleotides, wherein said oligonucleotide specifically hybridizes within an accessible region of TASK-3 mRNA, said region defined by nucleotides 99 through 112, 149 through 166, 197 through 207, 217 through 236, 290 through 300, 314 through 327, 448 through 462, 526 through 539, 710 through 742, 852 through 868, 896 through 910, 996 through 1028, 1042 through 1054, 1170 through 1183, or 1278 through 1296 of SEQ ID NO:1, and wherein said oligonucleotide inhibits the production of TASK-3.

2. A composition comprising the isolated antisense oligonucleotide of claim 1.

3. The composition of claim 2, wherein said composition comprises a plurality of isolated antisense oligonucleotides, wherein each antisense oligonucleotide specifically hybridizes within a different accessible region.

4. An isolated antisense oligonucleotide consisting essentially of 10 to 50 nucleotides, wherein said oligonucleotide specifically hybridizes within an accessible region of TASK-3 mRNA, said region defined by nucleotides 55 through 70, 101 through 156, 163 through 194, 226 through 240, 305 through 322, 434 through 443, 481 through 489, 500 through 512, 515 through 524, 540 through 557, 595 through 615, 641 through 658, 685 through 696, 700 through 711, 775 through 786, 791 through 806, 829 through 837, 929 through 947, 998 through 1013, 1088 through 1102, or 1108 through 1116 of SEQ ID NO:2, and wherein said isolated antisense oligonucleotide inhibits the production of TASK-3.

5. The isolated antisense oligonucleotide of claim 4, wherein said oligonucleotide comprises a modified backbone.

6. The isolated antisense oligonucleotide of claim 4, wherein said oligonucleotide comprises one or more non-natural internucleoside linkages.

7. The isolated antisense oligonucleotide of claim 4, wherein said oligonucleotide is an oligonucleotide analog.

8. The isolated antisense oligonucleotide of claim 4, wherein said oligonucleotide comprises one or more substituted sugar moieties.

9. The isolated antisense oligonucleotide of claim 4, wherein said oligonucleotide comprises nucleotide base modifications or nucleotide base substitutions.

10. A composition comprising the isolated antisense oligonucleotide of claim 4.

11. The composition of claim 10, wherein said composition comprises a plurality of isolated antisense oligonucleotides, wherein each antisense oligonucleotide specifically hybridizes within a different accessible region.

12. A nucleic acid construct comprising a regulatory element operably linked to a nucleic acid encoding a transcript, wherein said transcript specifically hybridizes within one or more accessible regions of TASK-3 mRNA in its native form.

13. A host cell comprising the nucleic acid construct of claim 12.

14. An isolated antisense oligonucleotide that specifically hybridizes within an accessible region of TASK-3 mRNA in its native form, wherein said antisense oligonucleotide inhibits production of TASK-3.

15. A method of decreasing production of TASK-3 in cells or tissues, comprising contacting said cells or tissues with an antisense oligonucleotide that specifically hybridizes within an accessible region of TASK-3.

16. A method for modulating pain in a mammal, said method comprising administering the isolated antisense oligonucleotide of claim 14 to said mammal.

17. A method of identifying a compound that modulates pain in a mammal, the method comprising:
contacting cells comprising a TASK-3 nucleic acid with a compound; and
detecting the amount of TASK-3 RNA or TASK-3 polypeptide in or secreted from said cell,
wherein a difference in the amount of TASK-3 RNA or TASK-3 polypeptide produced in the presence of said compound compared to the amount of TASK-3 RNA or TASK-3 polypeptide produced in the absence of said compound is an indication that said compound modulates pain in said mammal.

18. The method of claim 17, wherein the amount of said TASK-3 RNA is determined by Northern blotting.

19. The method of claim 17, wherein the amount of said TASK-3 polypeptide is determined by Western blotting.

20. The method of claim 17, wherein said compound is an antisense oligonucleotide that specifically hybridizes within an accessible region of TASK-3 mRNA in its native form, wherein said antisense oligonucleotide inhibits production of TASK-3.

21. A method for modulating pain in a mammal, said method comprising administering a compound to said mammal, wherein said compound modulates the expression of TASK-3.

22. The method of claim 21, wherein said compound is an antisense oligonucleotide that specifically hybridizes within an accessible region of TASK-3 mRNA in its native form, wherein said antisense oligonucleotide inhibits production of TASK-3.

23. The method of claim 21, wherein said pain is from diabetic neuropathy, postherpetic neuralgia, fibromyalgia, surgery, or chronic back pain.
